(19) United States
(12) Patent Application Publication
(10) Pub. No.: US 2025/0166747 A1
(43) Pub. Date: May 22, 2025

(54) AUTOMATED CLINICAL TRIAL MATCHING SYSTEM

(71) Applicant: GE Precision Healthcare LLC, Waukesha, WI (US)

(72) Inventors: Akshit Achara, Bengaluru (IN); Sanand Sasidharan, Bengaluru (IN); Anuradha Kanamarlapudi, Bengaluru (IN)

(21) Appl. No.: 18/952,925

(22) Filed: Nov. 19, 2024

(30) Foreign Application Priority Data
Nov. 21, 2023 (IN) 202341079184

(51) Int. Cl. G16H 10/20; G06F 40/20; G16H 10/60; G16H 70/20

(52) U.S. Cl. CPC G16H 10/20; G06F 40/20; G16H 10/60; G16H 70/20

(57) ABSTRACT

An automated clinical trial matching system is disclosed, which may be used by a care provider to generate a shortlist of clinical trials that a patient may participate in, or by a trial coordinator to generate a list of patients eligible for a given clinical trial. The clinical trial matching system may rely on large language models (LLMs), clinical ontologies, reference databases, clinical guidelines, etc., to generate patient data models and clinical trial data models including structured data that may be directly compared using a matching model. Clinical trials that match a patient and/or patients that match a clinical trial may then be ranked, with top ranking results being shortlisted for display to the care provider or trial coordinator. The clinical trial matching system may display the top ranking results in a graphical user interface that indicates elements of a clinical trial and patient data that match.

### TECHNICAL FIELD

Embodiments of the subject matter disclosed herein relate to matching patients with clinical trials that may be available to them, and vice-versa.

### BACKGROUND

When a patient has exhausted standard treatment options available via a health care system, a health care provider may recommend that the patient enroll in a clinical trial of a new or emerging medication that may not be approved for use within the health care system. The clinical trial may be conducted by a pharmaceutical company manufacturer of the new medication. However, finding a clinical trial that is suitable for the patient may be a laborious and time consuming process. The health care provider may be confronted with a large number of listings of potentially suitable clinical trials stored in a clinical trial database. The health care provider may review the listings one by one, comparing a textual description of eligibility criteria with the patient's health status.

Some attempts have been made to provide automated tools for matching patients to clinical trials. However, the automated tools may rely on comparing structured data extracted from patient records with structured data extracted from clinical trial listings. Because the structured patient data may include different entities and/or terminology than the structured clinical trial data, an accuracy of current automated tools may be low, where a number of suitable trials may not be detected by the current automated tools, at the same time many of the detected trials many not be suitable for the patient.

### SUMMARY

In one example, the current disclosure addresses the issues described above with method, comprising generating a patient data model from data of a patient extracted from an electronic health record (EHR); determining a disease state of the patient, based on the patient data model and clinical guidelines; generating a clinical trial data model from data of a clinical trial extracted from a database of clinical trials; comparing the patient data model with the clinical trial data model to determine whether the patient is a match for the clinical trial, based on the disease state, and inclusion and exclusion criteria of the clinical trial; and in response to determining that the patient is a match for the clinical trial, displaying one of the matching patient or the matching clinical trial on a display device.

The above advantages and other advantages, and features of the present description will be readily apparent from the following Detailed Description when taken alone or in connection with the accompanying drawings. It should be understood that the summary above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

Various aspects of this disclosure may be better understood upon reading the following detailed description and upon reference to the drawings in which:

FIG. 1 shows a block schematic diagram of an automated clinical trial matching system, in accordance with one or more embodiments of the present disclosure;

FIG. 2 shows a high-level block schematic diagram illustrating a workflow of the clinical trial matching system, in accordance with one or more embodiments of the present disclosure;

FIG. 3A shows a block schematic diagram of a first natural language processing module of the clinical trial matching system, in accordance with one or more embodiments of the present disclosure;

FIG. 3B shows a block schematic diagram of a second natural language processing module of the clinical trial matching system, in accordance with one or more embodiments of the present disclosure;

FIG. 4 is a flowchart showing an exemplary high-level method for generating a list of clinical trials available to a patient using the clinical trial matching system, in accordance with one or more embodiments of the present disclosure;

FIG. 5 is a flowchart showing an exemplary method for processing patient data to generate an enriched patient data model, in accordance with one or more embodiments of the present disclosure;

FIG. 6 is a flowchart showing an exemplary method for processing clinical data to generate an enriched clinical trial data model, in accordance with one or more embodiments of the present disclosure;

FIG. 7 is a flowchart showing an exemplary high-level method for generating a list of patients available to participate in a clinical trial using the clinical trial matching system, in accordance with one or more embodiments of the present disclosure;

FIG. 8 shows exemplary inclusion and exclusion criteria for a number of clinical trials, as prior art;

FIG. 9 shows an exemplary comparison between inclusion criteria of a clinical trial and clinical patient data, in accordance with one or more embodiments of the present disclosure; and

FIG. 10 is an image of an exemplary graphical user interface of the clinical trial matching system showing a match between a patient and a clinical trial, in accordance with one or more embodiments of the present disclosure.

The drawings illustrate specific aspects of the described systems and methods. Together with the following description, the drawings demonstrate and explain the structures, methods, and principles described herein. In the drawings, the size of components may be exaggerated or otherwise modified for clarity. Well-known structures, materials, or operations are not shown or described in detail to avoid obscuring aspects of the described components, systems and methods.

### DETAILED DESCRIPTION

An automated clinical trial matching system is provided herein, which may match patients with suitable clinical trials. The clinical trial matching system may generate a list of clinical trials for which a specific patient of a health care system may be eligible, or alternatively, a list of patients eligible to participate in a specific clinical trial, based on patient data stored in an electronic medical record (EMR) of the patient(s) and eligibility criteria of the clinical trial(s).

Clinical trials offer hope of cure or improvement to patients when other modes of treatment available to the patients are exhausted. A health care provider, such as an oncologist, may recommend one or more clinical trials to a patient. However, the administration of clinical trials falls under the purview of trial coordinators affiliated with pharmaceutical companies, who rely on different information systems. To generate a shortlist of suitable clinical trials, the health care provider may search in a clinical trial database. The health care provider may enter one or more terms into search tools to generate a list of clinical trials. The health care provider may review the trials on the list one by one to determine whether the patient meets eligibility criteria for each clinical trial. Determining whether the patient meets the eligibility criteria may take time, which may disrupt a workflow of the health care provider and reduce an amount of time that could be spent with patients.

Searching for an appropriate clinical trial may be further complicated by the fact that inclusion and/or exclusion criteria associated with a clinical trial may be phrased using a first set of terms that may not exactly match a second set of terms used to describe a patient's state of disease in a patient record of the patient. For example, the inclusion criteria may specify that the patient not be taking medications of a certain class. The patient record may include a list of medications taken by the patient, but not the class of medications. The inclusion criteria may specify that the patient be at a certain point in a patient healthcare journey, where whether the patient is at the certain point may have to be deduced based on events, test results, reports, etc. in the patient record. The reports and other patient data may include unstructured data (e.g., text), from which information may have to be extracted. As a result, current tools for finding clinical trials may generate inaccurate and/or incomplete results. Further, the task of finding one or more clinical trials suitable for a given patient may demand a high level of clinical experience and knowledge of the patient, making it difficult to delegate.

A similar problem is experienced by trial coordinators seeking to recruit eligible patients. Coordination between healthcare providers and the trial coordinators may result in a duplication of patient data, disease state and previous history that may lead to missed trials for a patient and poor recruitment into a trial.

To achieve better and more relevant trial matching and acceptance of trials by patients, an automated clinical trial matching system is disclosed, which may be used by a care provider to generate a shortlist of clinical trials that a patient may participate in that is more accurate and comprehensive than existing tools. The automated clinical trial matching system may also be used by a trial coordinator to generate a list of patients eligible for a given clinical trial. The clinical trial matching system may rely on various resources, including machine learning (ML) models, clinical ontologies, reference databases, clinical guidelines, etc., to generate patient data models and clinical trial data models including structured data that may be directly compared using a matching model, where a same vocabulary may be used to describe the trial criteria and the disease state of the patient. Clinical trials that match a patient and/or patients that match a clinical trial may then be ranked, with top ranking results being shortlisted for display to the care provider or trial coordinator. The clinical trial matching system may display the top ranking results in a graphical user interface that indicates elements of a clinical trial and patient data that match, to provide a degree of explainability with respect to how the results were ranked.

To achieve this, the clinical trial matching system may consult an entire longitudinal journey of the patient, use treatment guidelines to determine the disease state of the patient taking into consideration spatial as well as temporal conditions, match vocabulary of the patient model with clinical trial models using ontologies and knowledge graph representations, and apply complex reasoning to interpret trial criteria. The clinical trial matching system may benefit the patient care pathway in various ways. The system may provide precise and relevant matches of trials for the patient, considering the disease state, which may increase a recruitment rate for trials, reduce an amount of the time spent by the care provider identifying suitable clinical trials for the patient, and provide an end-to-end workflow for the patient.

The automated clinical trial matching system may rely on various models to determine matches, including one or more natural language processing (NLP) models for extracting structured data from text, as well as other AI models, such as machine learning (ML) or deep learning (DL) models, generative AI models, classification or prediction models, probabilistic models (e.g., Bayesian models), statistical models, decision tree models, or other types of models. For example, one or more of the various models may be implemented as a convolutional neural network (CNN) model including a plurality of hidden layers.

The clinical trial matching system may also rely on clinical guidelines used by the health care system, depending on a type of patient and/or pathology presented. For example, the National Comprehensive Cancer Network (NCCN) Clinical Practice Guidelines in Oncology (NCCN Guidelines®) may be a first set of clinical guidelines used for cancer patients. Additional sets of clinical guidelines may be used for specific types of cancer or other diseases. For example, for patients with prostate cancer, the Prostrate Imaging Reporting & Data System (PI-RADS®) may be used as a second set of clinical guidelines.

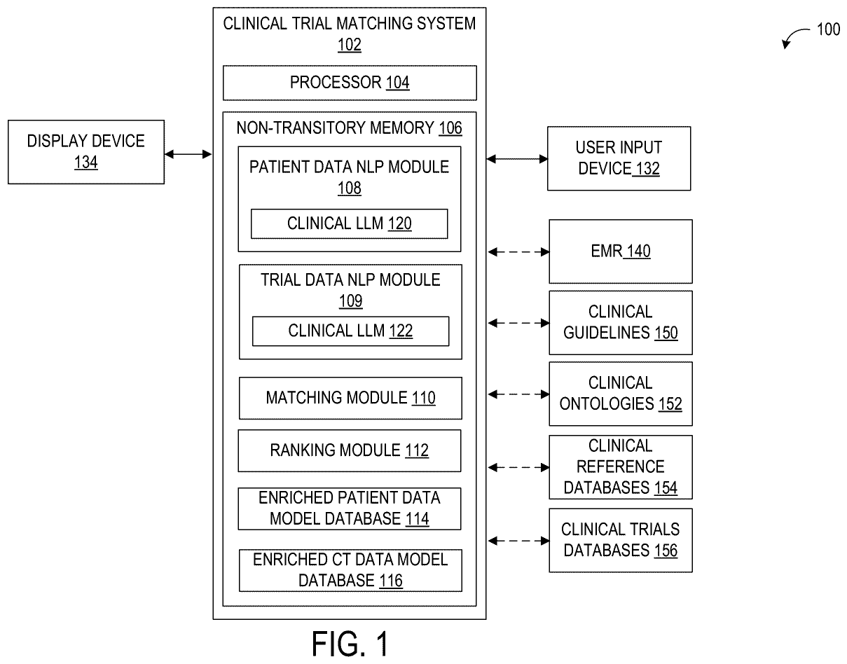

FIG. 1 shows a block diagram 100 of an exemplary automated clinical trial matching system 102, in accordance with an embodiment. Clinical trial matching system 102 may be used to match clinical trials with patients, as described above. In some embodiments, at least a portion of clinical trial matching system 102 is disposed at a device (e.g., workstation, edge device, server, etc.) communicably coupled to one or more healthcare and/or hospital networks and computer systems via wired and/or wireless connections, and can receive or access medical data (including patient data) stored in the one or more healthcare and/or hospital computer systems, such as an electronic medical record system (EMR) 140. The medical data may include online clinical reference materials, such as a set of digital clinical guidelines 150, which may be used by care providers to provide treatment alternatives to patients, one or more clinical ontologies 152, which may be used to harmonize vocabulary from different clinical sources, one or more clinical reference databases 154, and one or more clinical trial databases 156, which may store information about clinical trials being offered, including eligibility information.

Clinical trial matching system 102 may also be operably/communicatively coupled to a user input device 132 and a display device 134. In some examples, user input device 132 may be a shared input device of the one or more healthcare and/or hospital computer systems, and display device 134 may be a shared input device of the one or more healthcare and/or hospital computer systems.

Clinical trial matching system 102 includes a processor 104 configured to execute machine readable instructions stored in non-transitory memory 106. Processor 104 may be single core or multi-core, and the programs executed thereon may be configured for parallel or distributed processing. In some embodiments, processor 104 may optionally include individual components that are distributed throughout two or more devices, which may be remotely located and/or configured for coordinated processing. In some embodiments, one or more aspects of processor 104 may be virtualized and executed by remotely-accessible networked computing devices configured in a cloud computing configuration.

Non-transitory memory 106 may store at least a patient data NLP module 108, a trial data NLP module 109, a matching module 110, a ranking module 112, an enriched patient data model database 114, and an enriched clinical trial data model database 116. Patient data NLP module 108 may include at least one first clinical large language model (LLM) 120, and instructions for using first clinical LLM 120 to extract entities and relationships from unstructured (e.g., textual) patient data, such as reports. In various embodiments, first clinical LLM 120 may be a private implementation of a commercial, open source, or in-house LLM, such as OpenAI's GPT, which may be trained to extract entities and/or relationships from text data. Further, first clinical LLM 120 may be fine-tuned based on one or more private labeled clinical data sets. Trial data NLP module 109 may include at least one second clinical LLM 122, which may be trained to extract entities and relationships from unstructured clinical trial data, such as written inclusion/exclusion criteria. In some embodiments, second clinical LLM 122 may be the same LLM as first clinical LLM 120.

Matching module 110 may comprise instructions for comparing patient data models with clinical trial data models to determine a degree of similarity (e.g., whether a patient matches a clinical trial, based on structured patient data and structured clinical trial data). Ranking module 112 may comprise instructions for ranking results outputted by clinical trial matching system 102 (e.g., clinical trials for a given patient or patients for a given clinical trial), based on the comparisons performed by matching module 110.

Enriched patient data model database 114 may store a first plurality of patient data models generated from patient data, where the patient data may be retrieved and compiled from EMR 140. Enriched clinical trial data model database 116 may store a second plurality of clinical trial data models generated from clinical trial data, where the clinical trial data may be retrieved and compiled from the one or more clinical trial databases 156, for example.

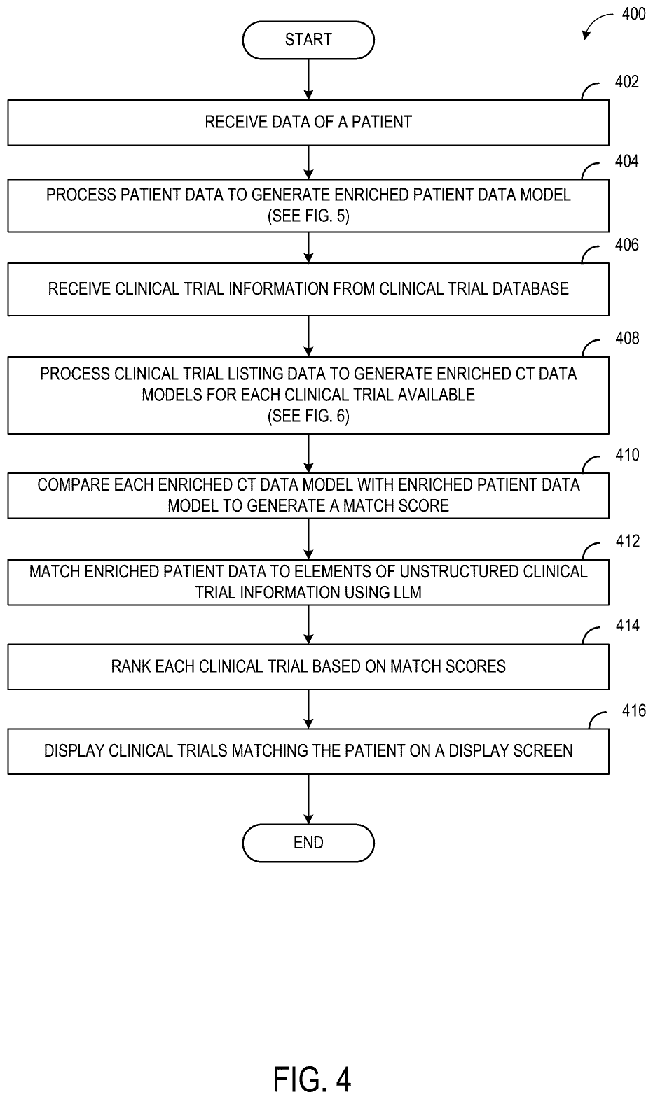

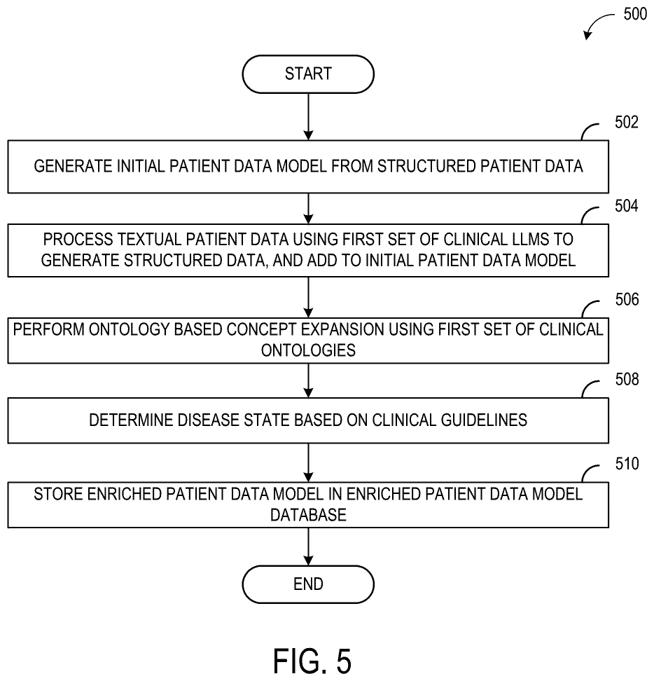

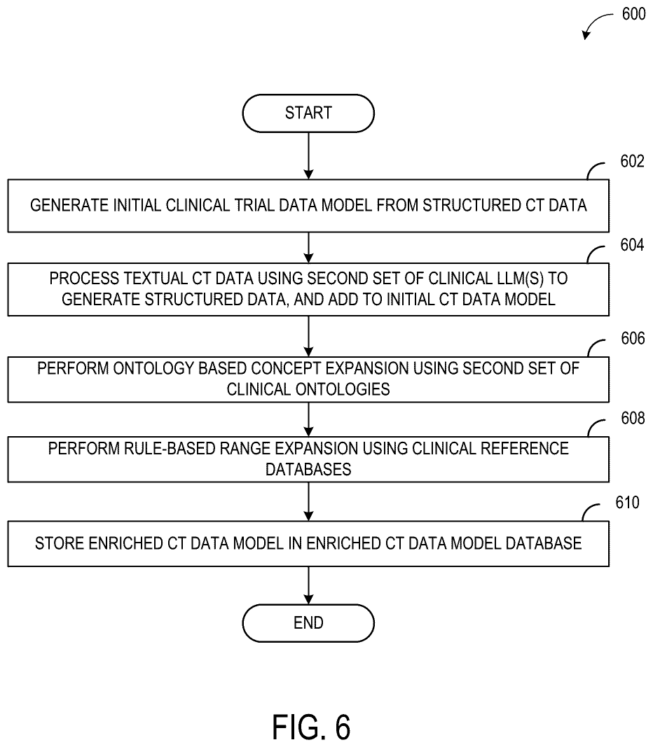

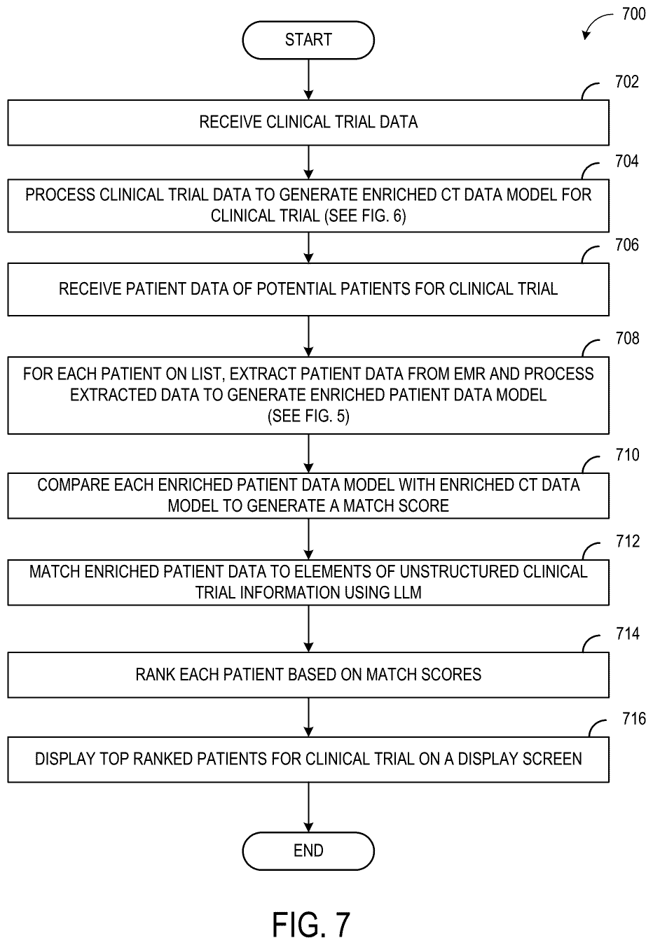

In particular, individually or in combination, patient data NLP module 108, trial data NLP module 109, matching module 110, and/or ranking module 112 may include instructions that, when executed by processor 104, cause clinical trial matching system 102 to conduct one or more of the steps of method 400 of FIG. 4, method 500 of FIG. 5, method 600 of FIG. 6, and/or method 700 of FIG. 7.

User input device 132 may comprise one or more of a touchscreen, a keyboard, a mouse, a trackpad, a microphone, a motion sensing camera, or other device configured to enable a user to interact with clinical trial matching system 102. Display device 134 may include one or more display devices utilizing virtually any type of technology. In some embodiments, display device 134 may comprise a computer monitor. Display device 134 may be combined with processor 104, non-transitory memory 106, and/or user input device 132 in a shared enclosure, or may be peripheral display devices and may comprise a monitor, touchscreen, projector, or other display device known in the art, which may enable a user to view responses to queries submitted to clinical trial matching system 102, and/or interact with various data stored in non-transitory memory 106.

It should be understood that clinical trial matching system 102 shown in FIG. 1 is for illustration, not for limitation. Another appropriate clinical trial matching system may include more, fewer, or different components.

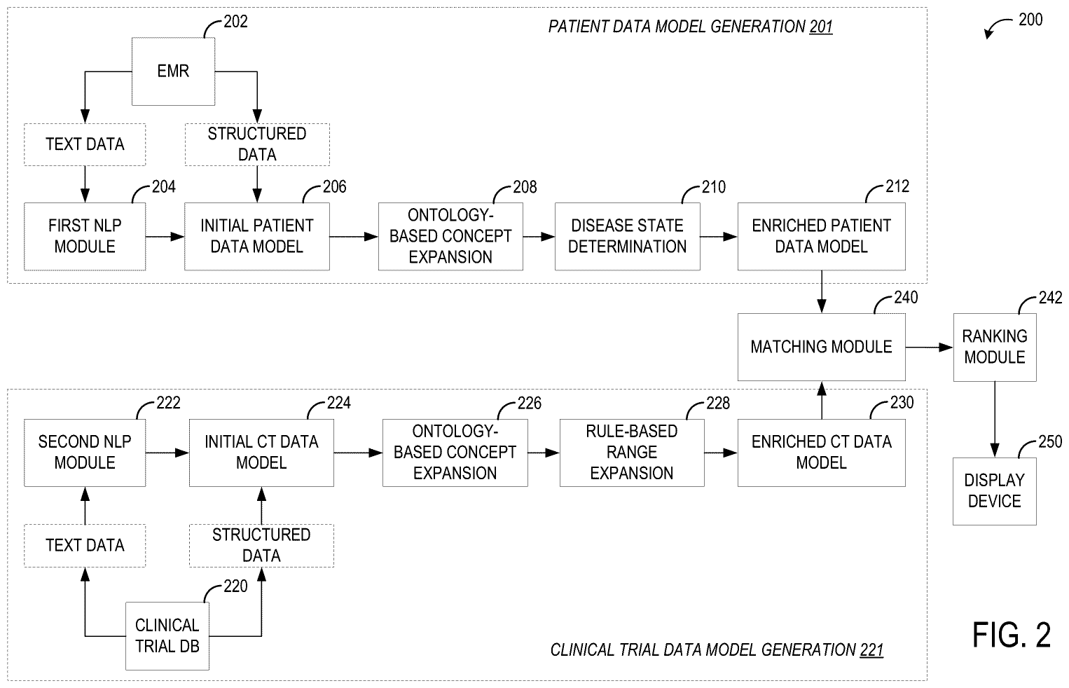

FIG. 2 shows a schematic diagram of a workflow 200 of a clinical trial matching system, which may be a non-limiting example of the clinical trial matching system of FIG. 1. A first method for matching clinical trials to a patient using the clinical trial matching system is described in reference to FIG. 4, which may follow workflow 200. A second method for matching patients to a clinical trial using the clinical trial matching system is described in reference to FIG. 7, which may also follow workflow 200.

Workflow 200 includes a patient data model generation block 201, during which an enriched patient data model may be generated for one or more patients of a healthcare system, and a clinical trial data model generation block 221, during which an enriched clinical trial data model may be generated for one or more clinical trials conducted by one or more pharmaceutical companies or other entities. The one or more enriched patient data models and the one or more enriched clinical trial data models may be compared using a matching module 240 (e.g., matching module 110 of FIG. 1) to generate a list of clinical trials that match a patient, or a list of patients that match a clinical trial. The list may then be ranked by a ranking module 242 for display on a display device 250, to be viewed by a care provider of the patient or a trial coordinator of the clinical trial, for example.

Patient data model generation block 201 and clinical trial data model generation block 221 may both include a series of similar steps, during which raw data in various formats is extracted and expanded to generate an enriched data model including structured data to facilitate a direct comparison. In other words, patient data model generation block 201 describes a first process for extracting patient data from medical records of the patient and constructing a patient data model, and clinical trial data model generation block 221 describes a second, similar process for extracting clinical trial data from one or more lists of clinical trials and constructing a clinical trial data model.

In patient data model generation block 201, the clinical trial matching system may extract patient data from a patient record stored in a hospital database, such as an EMR 202 (e.g., EMR 140). The patient data may include demographic data and clinical data. The clinical data may include ontology-linked data. Examples of ontology-linked data include Fast Healthcare Interoperability Resources (FHIR) data elements that are linked to specific nodes of an ontology, such as, for example, Logical Observation Identifiers Names and Codes (LOINC) or Systemized Nomenclature of Medicine-Clinical Terms (SNOMED), via a combination of a code system identifier (e.g., a uniform resource identifier (URI)) and a concept identifier (typically an alpha-numeric code unique to the ontology).

The patient data may also include structured data (e.g., labeled data or data stored in fields), and unstructured data (e.g., raw text including patient data, such as lab, test, or other reports, comments made by a care provider, etc.) The structured data may be extracted programmatically. A first NLP module 204 (e.g., patient data NLP module 108) may be used to convert the unstructured data into structured data, using a first set of one or more LLMs (e.g., clinical LLMs 120). The structured data outputted by first NLP module 204 may be combined with the extracted structured data to generate an initial patient data model 206.

Initial patient data model 206 may then be enriched via an ontology-based concept expansion process 208, where terms and vocabulary included in initial patient data model 206 are expanded using a first set of one or more relevant clinical ontologies (e.g., National Cancer Institute Thesaurus (NCIT), SNOMED, Unified Medical Language System (UMLS), etc.). Expanding the patient data may include, for example, expanding acronyms or abbreviations, including synonyms, converting vocabulary into commonly accepted terminology, and the like.

A disease state determination process 210 may be performed to precisely define a disease state of the patient. The disease state may be generated from and/or using one or more sets of clinical guidelines (e.g., clinical guidelines 150 of FIG. 1), which may be digital guidelines available online and/or on a computer system or network of a healthcare system, using the clinical trial matching system. The clinical guidelines may include different reference guidelines for different types of patients and/or pathologies. For example, a first set of clinical guidelines may relate to patients who have or are suspected of having cancer; a second set of clinical guidelines may relate to patients who have or are suspected of having an auto-immune disease; a third set of clinical guidelines may relate to patients who are suffering from traumatic wounds; and so on. In one example, the clinical guidelines include the NCCN Guidelines®. A result of the patient data model generation block may be the generation of an enriched patient data model 212, where enriched patient data model 212 includes highly structured patient data in a standardized format with broad coverage of synonymous terms.

In clinical trial data model generation block 221, the clinical trial matching system may extract clinical trial data from one or more clinical trial databases 220. The clinical trial data may include trial eligibility information, such as inclusion and exclusion criteria for a plurality of clinical trials. The clinical trial data may include structured data and unstructured data. The structured data may be extracted programmatically. A second NLP module 222 (e.g., trial data NLP module 109) may be used to convert the unstructured data into structured data, using a second set of one or more LLMs. The structured data outputted by second NLP module 222 may be combined with the extracted structured data to generate an initial clinical trial data model 224.

Initial clinical trial data model 224 may then be enriched via an ontology-based concept expansion process 226 similar to the ontology-based concept expansion process 208, where terms and vocabulary included in initial clinical trial data model 224 are expanded using a second set of one or more relevant clinical ontologies, which may be different from the first set of relevant clinical ontologies. For example, the second set of one or more relevant clinical ontologies may include the Medical Subject Headings (MeSH) ontology. Expanding the patient data may include, for example, expanding acronyms or abbreviations, including synonyms, converting vocabulary into commonly accepted terminology, and the like.

A rule-based range expansion process 228 may be performed to expand a range of values of inclusion and exclusion data (e.g., “Eastern Cooperative Oncology Group (ECOG) between 0 and 3”, or “American Joint Committee on Cancer (AJCC) Cancer Stages III to IV”), using one or more medical reference range databases of medical values (e.g., clinical reference databases 154). Expanding of value ranges may include, for example, obtaining allowed values of a parameter and their ordering from clinical reference databases (e.g., NCIT) and using that to convert the range expression to a list of all applicable values. For example, the implicitly defined range of “ECOG between 0 and 3” may be expanded to the explicit range “ECOG may take any of the values from the set {0,1,2,3}”, or “Cancer Stages III-IV” may be expanded to “Stages IIIA, IIIB, IIIC and IV”).

A result of the clinical trial data model generation block may be the generation of an enriched clinical trial data model 230, where enriched clinical trial data model 230 includes highly structured clinical trial data in the same standardized format as the enriched patient data model 212, with similar broad coverage of synonymous terms. Enriched clinical trial data model 230 may then be compared with enriched patient data model 212 by the matching module 240, to generate a list of matching clinical trials or patients.

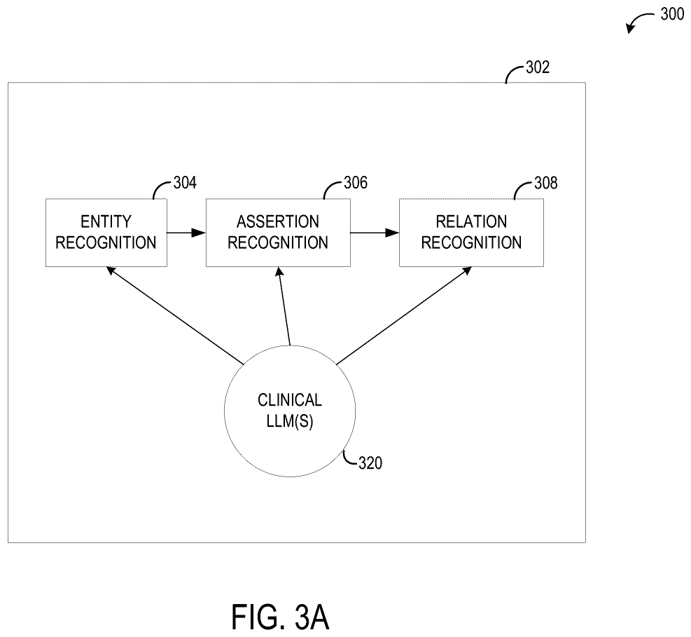

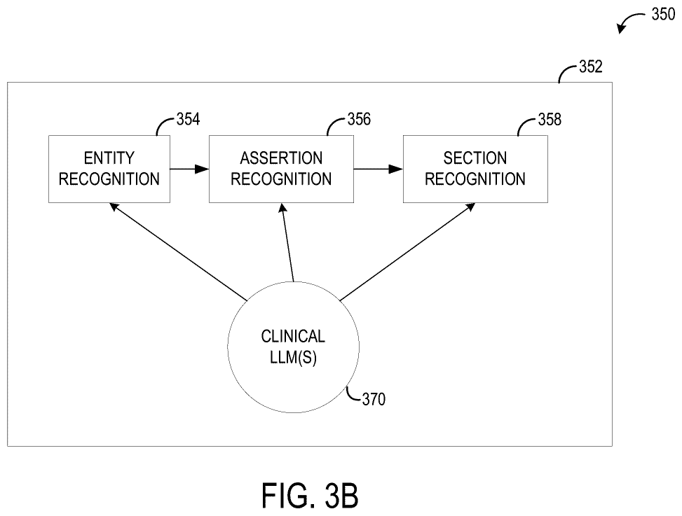

FIG. 3A shows a block schematic diagram 300 of a patient data NLP module 302, which may be a non-limiting example of first NLP module 204 of FIG. 2 and or patient data NLP module 108 of FIG. 1. Patient data NLP module 302 includes one or more clinical LLMs 320 (e.g., LLMs 120), which may be used to extract information from unstructured patient data, such as textual descriptions of a patient's diagnosis, disease state, written results of tests, labs, imaging studies, interventions, and/or treatments, care provider comments, and the like. Specifically, the one or more clinical LLMs 320 may perform an entity recognition task 304, where entities are recognized and extracted from the unstructured patient data. The entities may include words or phrases embedded in text that represent nouns, such as lesions, body parts, locations, diseases, etc. For example, the one or more clinical LLMs 320 may be trained to recognize a “nodular tumor extension” as a tumor; “mesorectal fat” as a body part including the tumor; and “8 to 10 o'clock” as a position of the tumor within the body part. The one or more clinical LLMs 320 may extract the entities, and output a set of labeled entities for inclusion in a patient data model.

The one or more clinical LLMs 320 may perform an assertion recognition task 306, where an existence of a specific type of entity such as a disease, tumor, condition, etc. may be recognized and asserted. For example, the one or more clinical LLMs 320 may be trained to recognize the entity “nodular tumor extension”, and assert (e.g., infer) that a tumor is present in the patient. The assertions may be included in the patient data model. The assertions may include logical inferences performed by the one or more clinical LLMs 320, logical deductions performed by the one or more clinical LLMs 320, or a logical correlation, etc.

The one or more clinical LLMs 320 may perform a relation recognition task 308, where relationships between entities are recognized and extracted from the unstructured patient data. For example, the one or more clinical LLMs 320 may be trained to recognize that the nodular tumor extension may be found within the mesorectal fat, at the 8 o'clock to 10 o'clock position. The relationships may be included in the patient data model.

FIG. 3B shows a block schematic diagram 350 of a clinical trial data NLP module 352, which may be a non-limiting example of second NLP module 222 of FIG. 2 and or trial NLP module 109 of FIG. 1. Clinical trial data NLP module 352 includes one or more clinical LLMs 370 (e.g., LLMs 122), which may be used to extract information from unstructured clinical trial data. Specifically, the one or more clinical LLMs 370 may perform one or more of an entity recognition task 354, which may be the same as or similar to the entity recognition task 304 of FIG. 3A; an assertion recognition task 306, which may be the same as or similar to the assertion recognition task 306 of FIG. 3A; and a section recognition task 358, where sections of the unstructured clinical trial data may be identified. For example, a beginning and end of Inclusion and Exclusion sections of the eligibly criteria may be identified.

Referring now to FIG. 4, an exemplary high-level method 400 is shown for determining a set of clinical trials available to a patient using a clinical trial matching system, such as clinical trial matching system 100 of FIG. 1. Method 400 may be carried out by a processor of the clinical trial matching system (e.g., processor 104), based on instructions stored in a memory of the clinical trial matching system (e.g., non-transitory memory 106). Method 400 may be performed in response to a command by a user of the clinical trial matching system, such as a care provider seeking to find one or more clinical trials suitable for the patient.

Method 400 begins at 402, where method 400 includes receiving data of a patient of a healthcare system. In various embodiments, the patient data may be received from a hospital network, for example, from an EMR such as EMR 140. The patient data may include demographic data (e.g., age, gender, ethnicity, etc.) and clinical data included in Electronic Health Records (EHRs) belonging to each patient, which may be ingested directly from hospital databases. The ingested data may be passed through a data ingestion pipeline. The ingested data may include any data containing a parameter that may be used for generating a match. This may include patient-provided data like age, gender and ethnicity during a registration process to a hospital. This may also include EMR and/or Digital Imaging and Communications in Medicine (DICOM) data from diagnostic procedures done at radiology and pathology departments, and/or notes from therapeutic procedures like radiotherapy or systemic therapies, as well as notes from a treating physician. Data not related to clinical procedures (e.g., billing or insurance data) may be excluded from the ingestion process.

At 404, method 400 includes processing the patient data to generate an enriched patient data model. The enriched patient data model may be a model of the patient data that includes structured data of the patient (e.g., as opposed to raw text data), where the structured data is represented in an expanded, standardized format. The enriched patient data model may be stored, for example, as an XML file using a markup language. In one example, the enriched patient data model may be stored as a Javascript Object Notation (JSON) file. Generating the enriched patient data model is described in greater detail below in reference to FIG. 5.

At 406, method 400 includes receiving clinical trial information from one or more clinical trial databases, such as ClinicalTrails.gov. In some embodiments, the clinical trial data may be ingested from the clinical trial databases on a periodic basis. The ingested data may then be processed through a data ingestion pipeline. The clinical trial data may include eligibility information, such as inclusion and exclusion criteria; timeline information, such as deadlines for participation, length of a trial, starting and ending dates; information about a medication that is the subject of the trial; and/or other information. The ingested clinical trial data may include any information that may be directly compared with the patient data described above, or information that may be used to rank recommended clinical trials according to a patient's preferences (e.g., such as distance to a trial facility, phase of a trial, etc.). A URL for a webpage of the trial may also be ingested and may be included in an output of the clinical trial matching system, in some examples. Clinical trial databases may be periodically (e.g., daily or weekly) checked for new or updated information, and re-ingestion may be performed when desirable.

At 408, method 400 includes processing the clinical trial data to extract data corresponding to a plurality of individual clinical trials, and generate a respective plurality of enriched clinical trial data models for the individual clinical trials. An enriched clinical trial model may be a model of clinical trial data associated with an individual clinical trial that includes structured data of the clinical trial (e.g., as opposed to raw text data), where the structured data is represented in a same expanded, standardized format as the enriched patient data model. To generate the enriched trial data model, text data included in clinical trial data may be analyzed and processed to be converted into structured data. Generating the enriched clinical trial data model is described in greater detail below in reference to FIG. 6.

At 410, method 400 includes comparing the enriched patient data model with the plurality of enriched clinical trial data models, to identify potential matches between the patient and clinical trials available to the patient. The comparing of the enriched patient data model with the plurality of enriched clinical trial data models may be performed by executing instructions stored in a matching module of the clinical trial matching system, such as matching module 110 of FIG. 1 and/or matching module 240 of FIG. 2. The enriched patient data model may be compared with the clinical trial data model to determine whether the patient is a match for the clinical trial based on the disease state, and inclusion and exclusion criteria of the clinical trial.

An enriched patient data model of the patient may be retrieved from the enriched patient data model database. Read parameters of the enriched patient data model may be used to create a search query for searching for matching enriched clinical trial data models in the enriched clinical trial model database. The expanded ranges of ordinal parameters and expanded concepts of categorical parameters may be used to perform the matching. In some embodiments, a relational database may be used to store the expanded ranges and concepts, and the matching may be performed by executing SQL queries on them, wherein the literal-values in the SQL where-clause corresponding to each relation are filled using corresponding values in the enriched patient data model for a given patient. In still other embodiments, a semantic knowledge-graph database may be used to store the expanded ranges and concepts, and matching may be performed by executing SPARQL queries on them, wherein the literal-values in the SPARQL where-clause corresponding to each relation are filled using corresponding values in the enriched patient data model for a given patient. It should be appreciated that the examples provided herein are for illustrative purposes, and a different method may be used to match the enriched patient data model with one or more enriched clinical trial data models without departing from the scope of this disclosure.

When an enriched clinical trial data model is compared to an enriched patient data model, the enriched clinical trial data model may be assigned a match score, which may represent a degree to which the enriched clinical trial data model matches the enriched patient data model. For example, the match score may be a normalized value between zero and one, where a zero indicates that the enriched clinical trial data model does not match the enriched patient data model (e.g., the clinical trial is not a match for the patient), and a one indicates that the enriched clinical trial data model is a perfect match for the enriched patient data model (e.g., the clinical trial is a perfect match for the patient). In one embodiment, each clinical trial is assigned a Boolean match score of either 0 or 1, where the trial gets the score of 0 if any of the match criteria is not satisfied, and a score of 1 if all the match criteria are satisfied.

At 412, method 400 includes matching elements of unstructured clinical trial information with the enriched patient data using an LLM. On some occasions, some of elements of unstructured data of the clinical trial information may not be converted to structured data and included in the enriched clinical trial data model, and may remain as unstructured text statements. This may happen, for example, when clinical trial criteria uses new vocabulary or grammar that the NLP module (e.g., trial data NLP module 109) cannot fully process. In such situations, part or all of the clinical trial criteria may be preserved in the original textual format, and the matching module may rely on an LLM to match the preserved unstructured data to the enriched patient data model. For example, the LLM may be second clinical LLM 122, or a different LLM.

At 414, method 400 includes ranking each individual clinical trial extracted from the clinical trial data, based on the match scores assigned to the respective clinical trial data models. In some embodiments, ranking the individual clinical trials may include reordering the individual clinical trials based on a descending order of the match scores. In other embodiments, the match scores may include various components, and the individual clinical trials may be reordered based on a weighting of the various components. For example, the clinical trials may be ranked according to various quality and practicality metrics, such as a phase of the study (e.g., later phases like phase 3 and phase 4 may be assigned a higher rank), a distance of a facility conducting the study from a location of the patient (e.g., closer facilities may be assigned a higher rank), etc. In still other embodiments, an artificial intelligence (AI) system, such as a rule-based system, may be used to reorder the individual clinical trials based on the match scores. In various embodiments, ranking the individual clinical trials may include generating a list of clinical trials that are matches for a patient, where the list excludes clinical trials that are not matches for the patient. For example, clinical trials having a match score above a threshold match score may be included in the list, and clinical trials having a match score less than the threshold match score may not be included in the list.

At 416, method 400 includes displaying clinical trials that are matches for the patient (e.g., above the threshold match score) on a display device. The display device may be a display device coupled to the clinical trial matching system, such as display device 134 of FIG. 1, or the display device may be a different display device. In one example, the display device may be a tablet or a smart phone. The clinical trials that are matches for the patient may be displayed within a graphical user interface (GUI) that shows how each clinical trial in the display matches the patient. In other words, the GUI may provide a degree of explainability with respect to how the match score was generated.

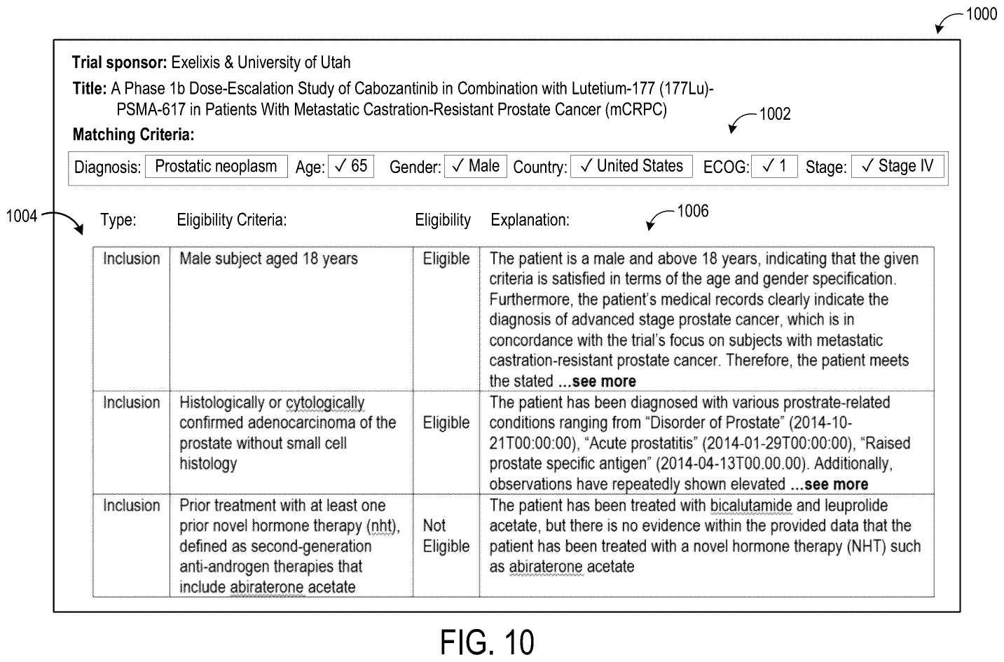

FIG. 10 shows an exemplary GUI 1000 of the clinical trial matching system. In the depicted embodiment, various criteria used to match the patient to a clinical trial are indicated in a table including a plurality of columns and rows. Trial criteria and corresponding (e.g., matching) patient data may be displayed side by side, to show how the patient meets each different individual trial criterion. Color coding may be used to indicate matching data, or data that does not match. For example, a first color may be used to show that a data element from patient data matches a corresponding data element from clinical trial data, a second color may be used to show that a data element from patient data does not match a corresponding data element from clinical trial data, and a third color may be used to show that a data element from patient data may match a corresponding data element from clinical trial data. Different data elements may be separated into different rows or columns to be easily distinguished.

For example, GUI 1000 shows a match between a patient and a clinical trial. A summary panel 1002 summarizes eligibility criteria of the clinical trial that are met by the patient. Additionally, an explainability panel 1004 shows a list of eligibility criteria, and for each criterion of the eligibility criteria, an indication of whether or not the patient meets the criterion, and a detailed explanation 1006 of how the system determined the patient's eligibility based on the criterion. The detailed explanation 1006 may be a 2-3 sentence description of how the match between patient data and the trial criteria was determined by the matching module. The explanation may include references to the source data which were used to arrive at the match. The detailed explanation 1006 may describe, for example, how patient information that was included in unstructured text matches with eligibility criteria extracted from the structured clinical trial data model. For example, explanation 1006 may reference a first textual description of the patient extracted from patient data, and a second textual description of a criterion included in the clinical trial, and show how the first textual description was mapped to the second textual description. As described above, the detailed explanations 1006 may be generated by one or more LLMs, such as clinical LLM 120 and/or clinical LLM 122. In this way, a user of GUI 1000 may review a “logic” applied by the clinical trial matching system, and verify whether inferences and/or deductions of the clinical trial matching system were correct and/or appropriate. By reviewing the logic, the user may verify the eligibility of the patient for the clinical trial.

Referring now to FIG. 5, an exemplary method 500 is shown for processing patient data to generate an enriched patient data model. Method 500 may be carried out as part of method 400 described above in reference to FIG. 4.

Method 500 begins at 502, where method 500 includes generating an initial patient data model from structured patient data included in the patient data received from the hospital network. The structured patient data may be included in various EHRs of the patient.

At 504, method 500 includes processing unstructured patient data (e.g., text data) using a first set of clinical LLMs (e.g., clinical LLMs 320 of FIG. 3A) to convert the unstructured patient data to structured patient data, at a patient data NLP module (e.g., patient data NLP module 108 of FIG. 1). The patient data NLP module parses textual information present in EHRs, such as consult notes, radiology findings, provider comments, etc., using the first set of clinical LLMs. The first set of clinical LLMs may include publically available clinical LLMs, such as BioBERT, PubMedBERT, etc. The first set of clinical LLMs may be trained on one or more general- and/or clinical-domain NLP data sets. The general-domain NLP data set may be a publicly available data set that may be used to train the clinical LLMs to respond to general questions in an intelligent manner. The clinical-domain NLP data set may be trained on medical and/or clinical data, such as clinical guidelines. Additionally, the first set of clinical LLMs may be fine-tuned using based on one or more private labeled clinical data sets. The clinical LLMs may extract entities, assertions, and/or relationships from the patient data, such as procedures undergone, drug-regimes administered, and diagnoses made. By fine-tuning the LLM on the one or more private labeled clinical data sets, an accuracy of the first set of clinical LLMs at extracting entities, assertions, relationships, and other concepts in the patient data may be increased. After the unstructured/textual data has been converted to structured data by the one or more LLMs, the converted structured data may be added to the initial patient data model.

At 506, method 500 includes performing an ontology-based concept expansion, using a first set of clinical ontologies. The first set of clinical ontologies may include, for example, NCIT, SNOMED, and/or a different clinical ontology. Specifically, the entities and other concepts extracted by the first set of clinical LLMs may be linked to unique nodes of the first set of clinical ontologies. Similar entities included at the unique nodes may be combined with the already structured and ontology-linked data of the initial patient data model created from the EHRs and results, as described above. The first set of clinical ontologies may be used to expand concepts into synonyms, non-abbreviated forms, and broader concepts. As an example, NSCLC may be expanded to ‘Non-small cell lung cancer’, as well as to the broader concepts of ‘Lung cancer’ and ‘Cancer’. As another example, the drug-regimen ‘Carboplatin’ may be expanded to the broader concept of ‘Platinum-based Chemotherapy Drug’ and ‘Chemotherapy Drug’.

At 508, method 500 includes determining a disease state of the patient based on the expanded initial patient data model and clinical guidelines (e.g., clinical guidelines 150 of FIG. 1). For example, a disease state determination module of the clinical trial matching system may determine all the diagnosis/treatment states the patient is currently in. In various embodiments, the determination may be made by comparing a longitudinal journey of the patient through diagnosis and treatment phases to a standard clinical guideline or care pathway (e.g., NCCN Guidelines, European Association of Urology (EAU) guidelines, etc.). For example, if the patient's prostate cancer has progressed while the patient is on anti-androgen medication, then the patient may be determined to be in a disease state called ‘castration-resistant prostate cancer’. Similarly, if the patient has not been yet exposed to a platinum-based chemotherapy drug, the patient may be determined to be in a disease state ‘platinum-naïve’. The purpose of this is to match to similar phrases appearing in trial inclusion/exclusion criteria when such trials seek patients with certain disease states. For each of the expanded concepts and inferred disease state, the source and the method of inferencing may be saved for later use for explainability purposes when displaying clinical trial matches on a display device. Once the disease state of the patient has been determined, the disease state may be included in the patient data model, which may now be referred to as an enriched patient data model.

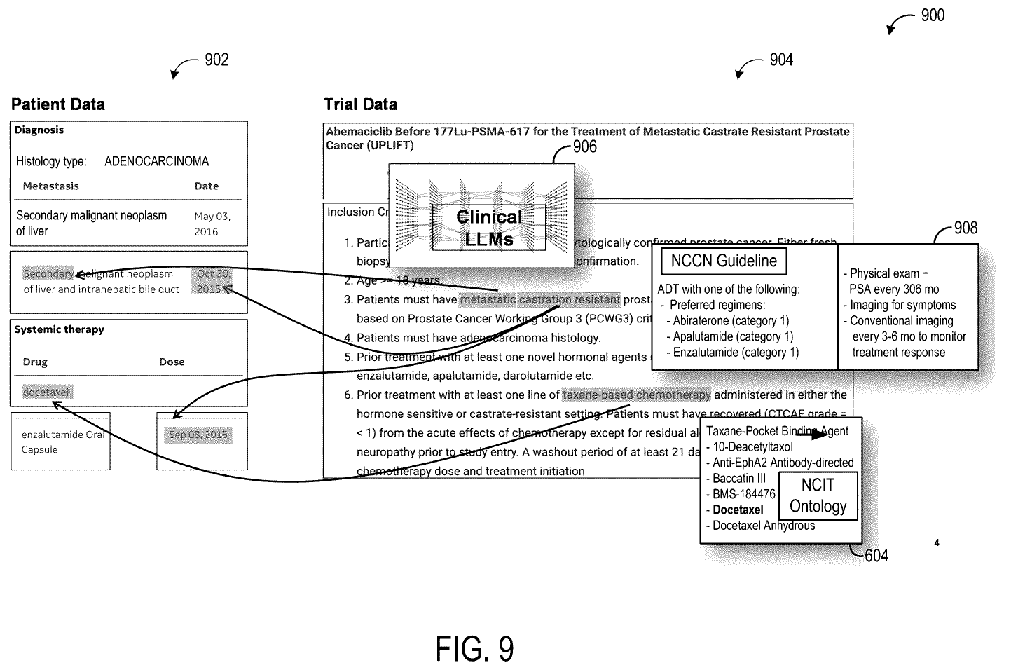

To understand the benefit of the clinical trial matching system, an example of how a longitudinal journey of the patient through diagnosis and treatment phases may be manually compared to a standard clinical guideline may be described in reference to FIG. 9. Referring to FIG. 9, a data comparison diagram 900 shows a representation of data of a patient 902 on the left, where the data may be included in an EHR. The patient data depicted in FIG. 9 includes a diagnosis and various clinical data which taken together, may indicate a disease state of the patient. A representation of data of a clinical trial 904 is shown on the right, where clinical trial data includes various inclusion criteria. The clinical trial data may be retrieved from a clinical trial database.

To determine whether clinical trial 904 is a match for patient 902, a care provider may identify the phrase “metastatic castration resistant” in clinical trial 904. To determine whether the word “metastatic” matches the disease state of the patient 902, the care provider may search the patient data of patient 902 for evidence of a progression of a tumor. The care provider may see that a malignant neoplasm of liver is described as “secondary”, and may infer that the criteria of “metastatic” has been met.

The care provider may then review the patient data to determine whether the patient data meets the criteria of “castration resistant”. To determine whether the patient meets the criteria of “castration resistant”, the care provider may determine whether patient 902 is taking androgen deprivation therapy (ADT). The care provider may see that patient 902 is taking enzalutamide. The care provider may consult the NCCN guidelines 908 and confirm that enzalutamide is listed as an ADT. The NCCN guidelines may further indicate that imaging is recommended every 3-6 months. The care provider may consult the patient data to determine a date when the enzalutamide was last administered. The care provider may see that the enzalutamide was last administered on Sep. 8, 2015. The care provider may then examine the patient data to determine whether the patient data includes a report indicating a progression of the tumor after that date. The care provider may see a report dated Oct. 20, 2015 indicating that a secondary malignant neoplasm of liver and intrahepatic bile duct was detected. As a result of the report, the care provider may infer that patient 902 is castration resistant.

The care provider may then see that the inclusion criteria states that patient 902 must have had “prior treatment with at least one line of taxane-based chemotherapy”. The care provider may then see that the patient is taking docetaxel, and may consult the NCIT ontology to verify that docetaxel is a taxane-based chemotherapy. Based on the patient meeting the condition of having metastatic castration resistant prostate cancer and having been treated with at least one line of taxane-based chemotherapy, the care provider may determine that clinical trial 904 is a match for patient 902.

Thus, the process of determining whether patient data of a patient (as represented in an EHR) matches eligibility criteria of a clinical trial may be time-consuming and laborious, where various elements of the patient data may be used to construct a timeline of events that may indicate whether a current disease state of the patient is described by the textual description of the clinical trial. In contrast, the proposed clinical trial matching system may more efficiently and quickly determine matches by using one or more clinical LLMs 906 to extract entities from both data representations (e.g., metastatic castration resistant prostate cancer, taxane-based chemotherapy, enzalutamide, secondary malignant neoplasm of liver, docetaxel), and advantageously use online resources such as clinical guidelines and medical/clinical ontologies to expand the entities to facilitate a direct comparison. For example, docetaxel may be expanded to a taxane-based chemotherapy; enzalutamide may be recognized as a class of ADT, and so on. Further, the LLMs may reconstruct a timeline of a patient treatment journey to estimate a current disease state referred to by the clinical trial. For example, if a patient's prostate cancer shows signs of progression while the patient is on ADT, the patient may be deemed provisionally as having a disease state of ‘castration-resistance prostate cancer’. The signs of progression of cancer may be in turn determined by making guideline-based inferences (e.g., using Response Evaluation Criteria in Solid Tumors (RECIST) criteria) on findings from radiology scans performed during the administration of ADT. The one or more clinical LLMs 906 may be used to make a determination of whether a radiology finding matches a certain RECIST criteria for cancer progression. Another example is the disease state of ‘taxane-naïve prostate cancer’, inferred by the absence of taxane-based chemotherapy drugs in the timeline of patient's treatment journey. By using the clinical trial matching system, an amount of time spent by the care provider may be significantly reduced, and a greater number of matches may be detected than may be found manually.

Returning to method 500, at 510, method 500 includes storing the enriched patient data model in an enriched patient data model database (e.g., enriched patient data model database 114 of FIG. 1), and method 500 ends.

Referring now to FIG. 6, an exemplary method 600 is shown for processing clinical trial data to generate an enriched clinical trial data model. Method 600 may be carried out as part of method 400 described above in reference to FIG. 4.

Method 600 begins at 602, where method 600 includes generating an initial clinical trial data model from structured clinical trial data included in the clinical trial data received from the clinical trial databases.

At 604, method 600 includes processing unstructured clinical trial data (e.g., text data) using a second set of clinical LLMs (e.g., clinical LLMs 370 of FIG. 3B) to convert the unstructured clinical trial data to structured clinical trial data, at a trial data NLP module (e.g., trial data NLP module 109 of FIG. 1). The trial data NLP module parses textual information present in the clinical trial data, such as demographic requirements, exclusion and inclusion criteria, etc., using the second set of clinical LLMs. The second set of clinical LLMs may include publically available clinical LLMs, such as BioBERT, PubMedBERT, etc. The second set of clinical LLMs may be trained on one or more general- and/or clinical-domain NLP data sets. In some embodiments, the second set of clinical LLMs may include one or more of the same clinical LLMs as the first set of clinical LLMs described above in reference to FIG. 5. In other embodiments, the second set of clinical LLMs may include different clinical LLMs as the first set of clinical LLMs. In some embodiments, the second set of clinical LLMs may not be fine-tuned using one or more private labeled clinical data sets.

The second set of clinical LLMs may extract entities, assertions, and/or sections from the clinical trial data. The trial data NLP module may parse inclusion and exclusion criteria and extract numerical or ordinal parameter ranges like age, cancer stage and ECOG performance status, for example. The trial data NLP module may also extract qualitative or categorical parameters like medications and co-morbidities that are included or excluded. After the unstructured/textual data has been converted to structured data by the second set of clinical LLMs, the converted structured data may be added to the initial clinical trial data model and combined with the other structured data.

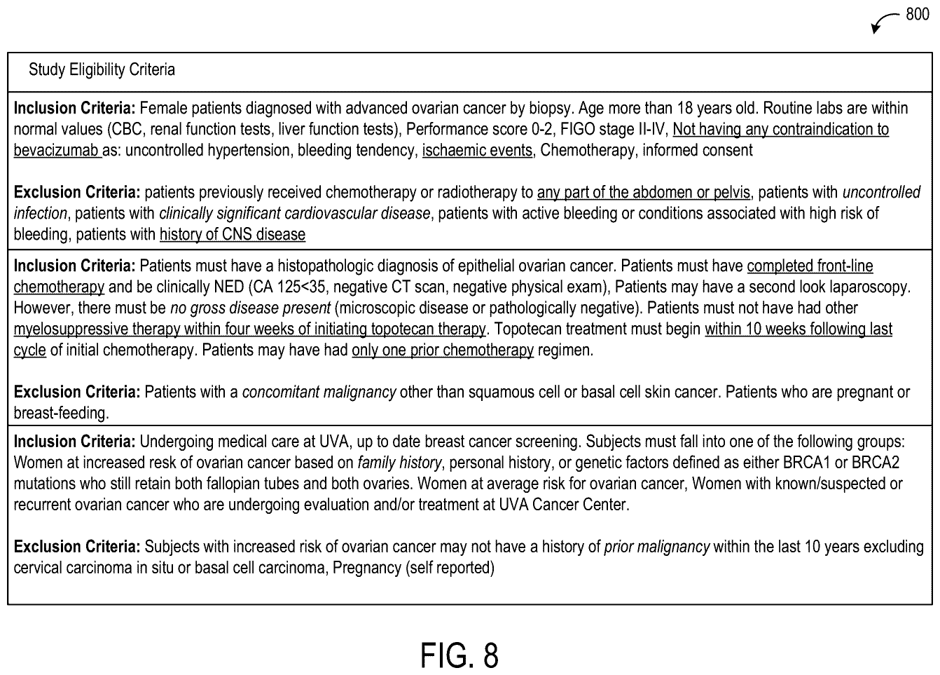

Turning briefly to FIG. 8, an excerpt 800 of clinical trial data is shown, where excerpt 800 includes various inclusion criteria and exclusion criteria of a plurality of clinical trials. Examples of entities that may be extracted from excerpt 800 by the second set of clinical LLMs are underlined. Examples of assertions that may be extracted from excerpt 800 are shown in italics.

Returning to method 600, at 606, method 600 includes performing an ontology-based concept expansion, using a second set of clinical ontologies. The second set of clinical ontologies may include one or more clinical ontologies of the first set of clinical ontologies, and/or different clinical ontologies. For example, the second set of clinical ontologies may include the MeSH ontology. Specifically, the entities and other concepts extracted from the clinical trial data by the second set of clinical LLMs may be linked to unique nodes of the second set of clinical ontologies to expand concepts into synonyms, non-abbreviated forms, and broader concepts, as described above in reference to method 500 of FIG. 5.

At 608, method 600 includes performing a rule-based range expansion using clinical reference databases. Specifically, a range expansion module may use a reference range database of medical values (e.g., ECOG, Cancer Stage, etc.) to expand the included and excluded range of values. For example, the implicitly defined range of “ECOG between 0 and 3”, which may appear as a phrase in the inclusion criteria of a trial, may be expanded to the explicit machine-readable range definition of “ECOG may take any of the values from the set {0,1,2,3}” by looking at the possible range of values and their order from a cancer-related reference database (e.g., NCIT). Similar method could be used to expand the range implicitly as “AJCC Cancer Stages III-IV” to “Stages IIIA, IIIB, IIIC and IV”. For each of the expanded concept and range, the source and the method of inferencing may be saved for later use for explainability purposes.

At 610, method 600 includes storing the enriched clinical trial data model in an enriched clinical trial data model database (e.g., enriched clinical trial data model database 116 of FIG. 1), and method 600 ends.

Turning now to FIG. 7, an exemplary high-level method for generating a list of patients available to participate in a clinical trial using a clinical trial matching system, such as clinical trial matching system 100 of FIG. 1. Method 700 may be carried out by a processor of the clinical trial matching system (e.g., processor 104), based on instructions stored in a memory of the clinical trial matching system (e.g., non-transitory memory 106). Method 700 may be performed in response to a command by a user of the clinical trial matching system, such as a trial coordinator seeking to recruit patients for a clinical trial.

Method 700 begins at 702, where method 700 includes receiving data of a clinical trial. In various embodiments, the clinical trial data may be may be extracted or received from a listing of trials in a clinical trials database (e.g., clinical trials databases 156), such as ClinicalTrials.gov. The patient data may include demographic criteria of subjects sought for a trial, as well as eligibility data such as inclusion and exclusion criteria for the trial, as shown in FIG. 8.

At 704, method 700 includes processing the clinical trial data to generate an enriched clinical trial data model. The enriched clinical trial data model may be a model of the clinical trial data that includes structured data of the clinical trial (e.g., as opposed to raw text data), where the structured data is represented in the same expanded, standardized format as the structured data of the enriched patient data model described above in reference to FIG. 4. The enriched clinical trial data model may be generated and stored as described in reference to FIG. 6.

At 706, method 700 includes receiving patient data of potential patients for the clinical trial. The patient data may be received from one or more hospital networks, as described above in reference to method 400 of FIG. 4. In some examples, the patient data may be ingested from hospital or health care databases on a periodic basis, and processed through the data ingestion pipeline described above. The patient data may include demographic data (e.g., age, gender, ethnicity, etc.) and clinical data included in Electronic Health Records (EHRs) belonging to each patient. The clinical data may include both structured and unstructured data describing a health status, disease state, condition, etc. of each patient.

At 708, method 700 includes processing the patient data to generate a plurality of enriched patient data models (e.g., an enriched patient data model for each patient of the patient data). The enriched patient data models may be generated as described above in reference to method 500 of FIG. 5.

At 710, method 700 includes comparing the enriched clinical trial data model with the plurality of enriched patient data models, to identify potential matches between the clinical trial and patients available for recruitment. The comparing of the enriched clinical trial data model with the plurality of enriched clinical trial data models may be performed by executing instructions stored in a matching module of the clinical trial matching system, such as matching module 110 of FIG. 1 and/or matching module 240 of FIG. 2. The comparing of the enriched clinical trial data model with the plurality of enriched clinical trial data models may be performed in a manner similar to that described above in reference to method 400. An enriched clinical trial data model of the patient may be retrieved from the enriched clinical trial data model database. Read parameters of the enriched clinical trial data model may be used to create a search query for searching for matching enriched patient data models in the enriched patient data model database. When an enriched clinical trial data model is compared to an enriched patient data model, the enriched patient data model may be assigned a match score, which may represent a degree to which the enriched patient data model matches the enriched clinical trial data model.

At 712, method 700 includes matching elements of unstructured clinical trial information with the enriched patient data using an LLM, in situations where elements of unstructured data of the clinical trial information are be converted to structured data and included in the enriched clinical trial data model. The matching module may rely on an LLM to match the preserved unstructured data to the enriched patient data model, such as second clinical LLM 122.

At 714, method 700 includes ranking each individual patient extracted from the patient data, based on the match scores assigned to the respective patient data models. The ranking of the individual patients may be performed as described above in reference to method 400.

At 716, method 700 includes displaying patients that are matches for the clinical trial (e.g., above the threshold match score) on a display device. The patients that are matches for the clinical trial may be displayed within a GUI that shows how each patient in the display matches the clinical trial. In other words, the GUI may provide a degree of explainability with respect to how the match score was generated, as described above in reference to FIG. 10. Method 700 may then end.

Thus, the proposed clinical trial matching system may support care providers by providing a way for patients and clinical trials to be matched that is less time consuming and more comprehensive than a manual process currently performed by care providers and trial coordinators. Rather than having to search and navigate through patient data or lists of clinical trials using a GUI, which may entail a sequence of actions including reading, selecting control elements of the GUI, typing information into the GUI, waiting for results, and then cross referencing data described using a first vocabulary with data stored using a second vocabulary, the matching may be performed in an automated manner with little operator input. By providing the care providers with a tool to match clinical trials and patients in a more efficient and faster manner, more patients may find treatment options and recruitment for trials may be increased, leading to a higher degree of positive outcomes.

The clinical trial matching system may provide clinically explainable matches, such that operators of the clinical trial matching system may see specific matching data without burdening clinicians with time-consuming and/or cumbersome interactions with a computerized system. As a result, the clinical trial matching system may reduce the digital device interactions of the care providers, freeing the care providers to devote more time with patients. An additional advantage of the proposed clinical trial matching system is that care providers and patients may be provided a greater degree of transparency with respect to how decisions are made about clinical trial participation and on what data the decisions are based, via the explanations included, for example, in the UI of FIG. 10. As a result, clinical trial matching system may facilitate a knowledgeable decision-making process for the patient and encourage a greater degree of trust.

The technical effect of using the clinical trial matching system to determine clinical trials that match patients or patients that match clinical trials is that a more comprehensive list of matching clinical trials or patients may be generated in a shorter amount of time in comparison to not only a manual process, but even another algorithm implemented in a processor that does not utilize the various aspects discussed herein. In this way, a use of computing resources of a health care system may be more efficiently managed and reduced. To elaborate, when a user of the clinical trial matching system matches a patient to a set of clinical trials, enriched clinical trial data models are generated and stored in the clinical trial data model database for each clinical trial referenced by the clinical trial matching system (in accordance with method 600), including clinical trials that do not match the patient. After the respective clinical trial data models are stored, the user, or a different user, can match a different patient to one or more clinical trials by comparing a patient data model of the different patient to the already-created and stored clinical trial data models, without having to consult data manually in the clinical trial database and/or without having to create or adjust the trial data models. That is, in an alternate scenario where the clinical trial matching system is not used, the user would have to log into the clinical trial database, search listings of clinical trials, and open each potentially matching clinical trial to review information of the clinical trial to determine whether a match exists. Each interaction with the clinical trial database consumes computing resources, including bandwidth, processing, and memory. Each interaction would have to be repeated for each patient, even for clinical trials that were previously consulted. And even if such actions were automated with a processor, the computer processing resources of such a system, for a given processor speed and memory capacity, would be more resource intensive than the approach outlined herein. In contrast to such technically inferior approaches, whether manual or automated with a processor, the disclosed clinical trial matching system converts the information included in the clinical trial database into a format (e.g., the clinical trial data model) that can be computationally compared with a patient data model based on a single instruction, with a substantially smaller consumption of the computing resources. As a result, once a clinical trial data model is generated, future comparisons of patients with clinical trials are facilitated at a lower computing cost as compared to other processor based approaches.

Similarly, when a user of the clinical trial matching system matches a clinical trial to a set of patients, enriched patient data models are generated and stored in the patient data model database for each patient referenced by the clinical trial matching system (in accordance with method 500). After the respective patient data models are stored, any user of the system can match a clinical trial to patients in the patient data model database by comparing a clinical trial data model of the different clinical trial to the already-created and stored patient data models, without having to retrieve patient data manually from one or more of various hospital databases and EMRs. This results in a decreased impact on computing resources (including processing and networking resources). In this way, an overall use of the computing resources of a health care system may be reduced. An up-front computing cost of generating the patient data models and clinical trial data models is more than offset by a reduction in the use of the computing resources spent on patients and clinical trials already stored on the clinical trial matching system.

The disclosure also provides support for a method, comprising: generating a patient data model from data of a patient extracted from an electronic health record (EHR), determining a disease state of the patient, based on the patient data model and clinical guidelines, generating a clinical trial data model from data of a clinical trial extracted from a database of clinical trials, comparing the patient data model with the clinical trial data model to determine whether the patient is a match for the clinical trial, based on the disease state, and inclusion and exclusion criteria of the clinical trial, and in response to determining that the patient is a match for the clinical trial, displaying one of the matching patient or the matching clinical trial on a display device. In a first example of the method, generating the patient data model further comprises using a first large language model (LLM) to extract entities, make assertions about entities, and recognize relationships between entities in unstructured patient data extracted from the EHR. In a second example of the method, optionally including the first example, the unstructured patient data includes textual descriptions of one or more of a diagnosis, a disease state, written results of tests, labs, imaging studies, a treatment, and a care provider comment. In a third example of the method, optionally including one or both of the first and second examples, generating the patient data model further comprises expanding entities extracted from the patient data to include synonyms, non-abbreviated forms, and broader concepts, using a clinical ontology. In a fourth example of the method, optionally including one or more or each of the first through third examples, generating the clinical trial data model further comprises using a second large language model (LLM) to extract entities from unstructured clinical trial data and identify beginnings and endings of inclusion and exclusion sections of eligibly criteria in the unstructured clinical trial data. In a fifth example of the method, optionally including one or more or each of the first through fourth examples, determining the disease state of the patient based on the patient data model and the clinical guidelines further comprises comparing a longitudinal journey of the patient through various diagnosis and treatment phases to a standard clinical guideline. In a sixth example of the method, optionally including one or more or each of the first through fifth examples, comparing the patient data model with the clinical trial data model to determine whether the patient is a match for the clinical trial further comprises calculating a match score based on a similarity between the patient data model and the clinical trial data model, and determine that the patient is a match for the clinical trial in response to the match score being greater than a threshold match score. In a seventh example of the method, optionally including one or more or each of the first through sixth examples, displaying the matching clinical trial on the display device further comprises displaying a plurality of matching clinical trials on the display device, the plurality of matching clinical trials ranked based on the match score of each clinical trial of the plurality of matching clinical trials. In a eighth example of the method, optionally including one or more or each of the first through seventh examples, the method further comprises: displaying the plurality of matching clinical trials in a graphical user interface (GUI), wherein the GUI includes a display of an explanation of how a criterion of a clinical trial matches with the patient data, the explanation referencing a first textual description of a patient condition extracted from the data of the patient, and a second textual description of the criterion, and explaining how the first textual description was mapped to the second textual description. In a ninth example of the method, optionally including one or more or each of the first through eighth examples, the method is applied to generate a list of clinical trials for which a selected patient is eligible. In a tenth example of the method, optionally including one or more or each of the first through ninth examples, the method is applied to generate a list of patients that may be eligible for a selected clinical trial.

The disclosure also provides support for a clinical trial matching system, comprising: one or more large language models (LLM), a processor, and a memory storing instructions that when executed, cause the processor to: determine whether a clinical trial is a match for a patient by comparing a first patient data model of the patient with a second clinical trial data model of the clinical trial, the first patient data model generated from patient data using a first LLM of the one or more LLMs, the second clinical trial data model generated from clinical trial data retrieved from a clinical trials database using a second LLM of the one or more LLMs, and in response to determining that the clinical trial is a match for the patient: indicate that the clinical trial is a match for the patient on a display device, and display data elements of the first patient data model that match with clinical trial criteria of the second clinical trial data model in a graphical user interface (GUI) on the display device. In a first example of the system, further instructions are stored in the memory that when executed, cause the processor to expand concepts included in the first patient data model and/or the second clinical trial data model using one or more clinical ontologies. In a second example of the system, optionally including the first example, further instructions are stored in the memory that when executed, cause the processor to store the expanded concepts in a relational database, and compare the first patient data model with the second clinical trial data model by executing Structured Query Language (SQL) queries on the stored concepts, wherein portions of the SQL queries are filled in using corresponding values of the patient data model. In a third example of the system, optionally including one or both of the first and second examples, further instructions are stored in the memory that when executed, cause the processor to store the expanded concepts in a semantic knowledge-graph database, and compare the first patient data model with the second clinical trial data model by executing SPARQL Protocol and RDF Query Language (SPARQL) queries on the stored expanded concepts, wherein portions of the SPARQL queries are filled in using corresponding values of the patient data model. In a fourth example of the system, optionally including one or more or each of the first through third examples, further instructions are stored in the memory that when executed, cause the processor to display in the GUI, for a data element of the first patient data model that matches a criterion of the second clinical trial data model, an explanation of how the criterion matches the data element, the explanation including a mapping of a first textual description of the data element extracted from the data of the patient with a second textual description of the criterion extracted from the clinical trial data. In a fifth example of the system, optionally including one or more or each of the first through fourth examples, the first LLM is the same as the second LLM. In a sixth example of the system, optionally including one or more or each of the first through fifth examples: the first LLM is a clinical LLM trained to extract information from unstructured patient data including textual descriptions of one or more of a patient's diagnosis, disease state, written results of tests, labs, imaging studies, interventions, and/or treatments, and care provider comments, and perform inferences based on the extracted information, and the second LLM is trained to extract information from unstructured clinical trial data, the extracted information including a beginning and an end of an inclusion section of eligibility criteria of the unstructured clinical trial data, and a beginning and an end of an exclusion section of the eligibility criteria.

The disclosure also provides support for a method, comprising: generating a clinical trial data model from data of a clinical trial extracted from a database of clinical trials, generating a plurality of patient data models from patient data of a respective plurality of patients extracted from a hospital database, comparing the clinical trial data model with each patient data model of the plurality of patient data models to determine a set of patients that match the clinical trial, and displaying the set of patients that match the clinical trial on a display device. In a first example of the method, displaying the set of patients that match the clinical trial on the display device further comprises displaying, in a graphical user interface (GUI), for a patient of the set of patients, an explanation of how the patient data of the patient matches an eligibility criterion of the clinical trial, the explanation including a mapping of a first textual description of an element of the patient data to a second textual description of the criterion extracted from the clinical trial data model.

When introducing elements of various embodiments of the present disclosure, the articles “a,” “an,” and “the” are intended to mean that there are one or more of the elements. The terms “first,” “second,” and the like, do not denote any order, quantity, or importance, but rather are used to distinguish one element from another. The terms “comprising,” “including,” and “having” are intended to be inclusive and mean that there may be additional elements other than the listed elements. As the terms “connected to,” “coupled to,” etc. are used herein, one object (e.g., a material, element, structure, member, etc.) can be connected to or coupled to another object regardless of whether the one object is directly connected or coupled to the other object or whether there are one or more intervening objects between the one object and the other object. In addition, it should be understood that references to “one embodiment” or “an embodiment” of the present disclosure are not intended to be interpreted as excluding the existence of additional embodiments that also incorporate the recited features.

In addition to any previously indicated modification, numerous other variations and alternative arrangements may be devised by those skilled in the art without departing from the spirit and scope of this description, and appended claims are intended to cover such modifications and arrangements. Thus, while the information has been described above with particularity and detail in connection with what is presently deemed to be the most practical and preferred aspects, it will be apparent to those of ordinary skill in the art that numerous modifications, including, but not limited to, form, function, manner of operation and use may be made without departing from the principles and concepts set forth herein. Also, as used herein, the examples and embodiments, in all respects, are meant to be illustrative and should not be construed to be limiting in any manner.

1. A method, comprising:
generating a patient data model from data of a patient extracted from an electronic health record (EHR);
determining a disease state of the patient, based on the patient data model and clinical guidelines;
generating a clinical trial data model from data of a clinical trial extracted from a database of clinical trials;
comparing the patient data model with the clinical trial data model to determine whether the patient is a match for the clinical trial, based on the disease state, and inclusion and exclusion criteria of the clinical trial; and
in response to determining that the patient is a match for the clinical trial, displaying one of the matching patient or the matching clinical trial on a display device.

2. The method of claim 1, wherein generating the patient data model further comprises using a first large language model (LLM) to extract entities, make assertions about entities, and recognize relationships between entities in unstructured patient data extracted from the EHR.

3. The method of claim 2, wherein the unstructured patient data includes textual descriptions of one or more of a diagnosis, a disease state, written results of tests, labs, imaging studies, a treatment, and a care provider comment.

4. The method of claim 2, wherein generating the patient data model further comprises expanding entities extracted from the patient data to include synonyms, non-abbreviated forms, and broader concepts, using a clinical ontology.

5. The method of claim 1, wherein generating the clinical trial data model further comprises using a second large language model (LLM) to extract entities from unstructured clinical trial data and identify beginnings and endings of inclusion and exclusion sections of eligibly criteria in the unstructured clinical trial data.

6. The method of claim 1, wherein determining the disease state of the patient based on the patient data model and the clinical guidelines further comprises comparing a longitudinal journey of the patient through various diagnosis and treatment phases to a standard clinical guideline.

7. The method of claim 1, wherein comparing the patient data model with the clinical trial data model to determine whether the patient is a match for the clinical trial further comprises calculating a match score based on a similarity between the patient data model and the clinical trial data model, and determine that the patient is a match for the clinical trial in response to the match score being greater than a threshold match score.

8. The method of claim 7, wherein displaying the matching clinical trial on the display device further comprises displaying a plurality of matching clinical trials on the display device, the plurality of matching clinical trials ranked based on the match score of each clinical trial of the plurality of matching clinical trials.

9. The method of claim 8, further comprising displaying the plurality of matching clinical trials in a graphical user interface (GUI), wherein the GUI includes a display of an explanation of how a criterion of a clinical trial matches with the patient data, the explanation referencing a first textual description of a patient condition extracted from the data of the patient, and a second textual description of the criterion, and explaining how the first textual description was mapped to the second textual description.

10. The method of claim 1, wherein the method is applied to generate a list of clinical trials for which a selected patient is eligible.

11. The method of claim 1, wherein the method is applied to generate a list of patients that may be eligible for a selected clinical trial.

12. A clinical trial matching system, comprising:
one or more large language models (LLM);
a processor, and a memory storing instructions that when executed, cause the processor to:
determine whether a clinical trial is a match for a patient by comparing a first patient data model of the patient with a second clinical trial data model of the clinical trial, the first patient data model generated from patient data using a first LLM of the one or more LLMs, the second clinical trial data model generated from clinical trial data retrieved from a clinical trials database using a second LLM of the one or more LLMs; and
in response to determining that the clinical trial is a match for the patient:
indicate that the clinical trial is a match for the patient on a display device; and
display data elements of the first patient data model that match with clinical trial criteria of the second clinical trial data model in a graphical user interface (GUI) on the display device.

13. The clinical trial matching system of claim 12, wherein further instructions are stored in the memory that when executed, cause the processor to expand concepts included in the first patient data model and/or the second clinical trial data model using one or more clinical ontologies.

14. The clinical trial matching system of claim 13, wherein further instructions are stored in the memory that when executed, cause the processor to store the expanded concepts in a relational database, and compare the first patient data model with the second clinical trial data model by executing Structured Query Language (SQL) queries on the stored concepts, wherein portions of the SQL queries are filled in using corresponding values of the patient data model.

15. The clinical trial matching system of claim 14, wherein further instructions are stored in the memory that when executed, cause the processor to store the expanded concepts in a semantic knowledge-graph database, and compare the first patient data model with the second clinical trial data model by executing SPARQL Protocol and RDF Query Language (SPARQL) queries on the stored expanded concepts, wherein portions of the SPARQL queries are filled in using corresponding values of the patient data model.

16. The clinical trial matching system of claim 12, wherein further instructions are stored in the memory that when executed, cause the processor to display in the GUI, for a data element of the first patient data model that matches a criterion of the second clinical trial data model, an explanation of how the criterion matches the data element, the explanation including a mapping of a first textual description of the data element extracted from the data of the patient with a second textual description of the criterion extracted from the clinical trial data.

17. The clinical trial matching system of claim 12, wherein the first LLM is the same as the second LLM.

18. The clinical trial matching system of claim 12, wherein:
the first LLM is a clinical LLM trained to extract information from unstructured patient data including textual descriptions of one or more of a patient's diagnosis, disease state, written results of tests, labs, imaging studies, interventions, and/or treatments, and care provider comments, and perform inferences based on the extracted information; and
the second LLM is trained to extract information from unstructured clinical trial data, the extracted information including a beginning and an end of an inclusion section of eligibility criteria of the unstructured clinical trial data, and a beginning and an end of an exclusion section of the eligibility criteria.

19. A method, comprising:
generating a clinical trial data model from data of a clinical trial extracted from a database of clinical trials;
generating a plurality of patient data models from patient data of a respective plurality of patients extracted from a hospital database;
comparing the clinical trial data model with each patient data model of the plurality of patient data models to determine a set of patients that match the clinical trial; and
displaying the set of patients that match the clinical trial on a display device.

20. The method of claim 19, wherein displaying the set of patients that match the clinical trial on the display device further comprises displaying, in a graphical user interface (GUI), for a patient of the set of patients, an explanation of how the patient data of the patient matches an eligibility criterion of the clinical trial, the explanation including a mapping of a first textual description of an element of the patient data to a second textual description of the criterion extracted from the clinical trial data model.
